(19) United States
(12) Patent Application Publication
(10) Pub. No.: US 2022/0218929 A1
(43) Pub. Date: Jul. 14, 2022

(54) TUBE INTRODUCER INTUBATION DEVICE

(71) Applicant: Wolf Technical Services, Inc., Fishers, IN (US)

(72) Inventors: Zach Wagner, Cicero, IN (US); Melissa Montgomery, Alexandria, VA (US); Jarrett Waters, Camby, IN (US); Aaron James Tolly, Fishers, IN (US)

(21) Appl. No.: 17/610,936

(22) PCT Filed: Jun. 2, 2020
(86) PCT No.: PCT/US2020/035739

§ 371 Date: Nov. 12, 2021

Related U.S. Application Data
(60) Provisional application No. 62/856,103, filed on Jun. 2, 2019.

(51) Int. Cl. A61M 16/04

(52) U.S. Cl. CPC A61M 16/0488

(57) ABSTRACT

An endotracheal tube introducer that includes a controller, a flexible tip, and a tube guide. The controller has an actuator and a rotatable bearing. The actuator operably coupled to move the rotatable bearing. The tube guide is operably coupled between the controller and the flexible tip. The flexible tip is operably coupled to the rotatable bearing to move in at least two degrees of freedom responsive to movement of the actuator.

This application claims the benefit of U.S. Provisional Patent Application Ser. No. 62/856,103, filed Jun. 2, 2019.

### FIELD OF THE INVENTION

The present invention is directed to methods and apparatus for performing oral intubation.

### BACKGROUND

Various medical treatments require assisted respiration, for example, from a ventilator. By way of non-limiting surgical anesthesia, illness affecting respiratory function, and trauma can all require assisted respiration. Assisted respiration is carried out through a tube inserted through the mouth of the patient and directed to the larynx. The process of inserting the tube into position for respiration is known as oral intubation.

In some patients, the process of intubation is complicated by anatomical conditions, known in the art as difficult airways. To intubate a patient with a difficult airway, it is necessary for the medical professional to be able to navigate through any obstructions in the mouth and at the rear of the throat to successfully place a tube to establish an airway.

A difficult airway can present risk of intubation failure, which can be life threatening, or can result in injury due to the urgency to complete the intubation. Many intubations involve the use of video laryngoscopy, which involves a specialized imaging device designed to provide video images of the patient's airway to assist the physician in the intubation procedure. Other methods and devices have been used in conjunction with the video laryngoscope, and some that use other imaging and guiding techniques, to improve the intubation procedure.

In many prior art devices, the airway tube is guided into the airway using an introducer or stylet. In one prior art device, an introducer mechanism has an actuating tip. This device, a version of which is shown in U.S Patent Publication no. 2017/0203075, bends in a single direction, providing one degree of freedom movement of the actuating tip of the introducer.

An alternative to the inexpensive, disposable options is using a bronchoscope to hold and transport the tracheal tube and a video scope to view the tissue on the way through the patient's throat. Unfortunately, cleaning the bronchoscope after each use or replacing a broken device is prohibitively expensive.

### SUMMARY

At least one embodiment described herein resolve the issues of prior art solutions to intubating difficult airways using a multi-dimensionally articulable introducer. The introducer eases placement of the tracheal tube when performing oral intubation in difficult airways.

A first embodiment is an endotracheal tube introducer that includes a controller, a flexible tip, and a tube guide. The controller has an actuator and a rotatable bearing. The actuator is operably coupled to move the rotatable bearing. The tube guide is operably coupled between the controller and the flexible tip. The flexible tip is operably coupled to the rotatable bearing to move in at least two degrees of freedom responsive to movement of the actuator.

Another aspect of the invention is a novel intubation method using the multi-dimensional articulable introducer or stylet. Once the introducer unit has been steered into place beyond the vocal cords with the assistance of the actuator and flexible tip, the tracheal tube can be slid off of the guide tube and into place in the airway. The device simplifies locating the tube with enhanced range of motion maneuverability through the airway. The steering intubation device decreases the likelihood of injuries and mortalities associated with intubation of difficult airways.

The above-described features and advantages, as well as others, will become more readily apparent to those of ordinary skill in the art by reference to the following detailed description and accompanying drawings

### BRIEF DESCRIPTION OF THE DRAWINGS

FIG. 1 shows a perspective view of a first embodiment of an articulable stylet or introducer;

FIG. 2 shows a side plan view of the introducer of FIG. 1;

FIG. 3 shows a fragmentary perspective view of the tube guide and flexible tip of the introducer of FIG. 1 illustrating one aspect of the movement of the flexible tip;

FIG. 4 shows a side plan view of the controller of the introducer of FIG. 1;

FIG. 5 shows a perspective view of the controller of FIG. 4 with one of the side covers removed;

FIG. 6 shows an exploded perspective view of the controller of FIG. 4;

FIG. 7 shows an exploded perspective view the flexible tip of the introducer of FIG. 1;

FIG. 7A shows a fragmentary perspective view of the nub of the flexible tip with wire segments assembled thereon;

FIG. 8 shows a perspective view of the introducer with an endotracheal tube disposed thereon;

FIG. 9 shows a flow diagram of an exemplary method of using the introducer of FIG. 1;

FIG. 10 shows a fragmentary plan view of the introducer with an endotracheal tube affixed thereto;

FIG. 11 shows a fragmentary plan view of the introducer with the endotracheal tube partially removed; and

FIG. 12 shows a plan view of an alternative embodiment of a controller that may be used in the introducer of FIG. 1.

### DETAILED DESCRIPTION

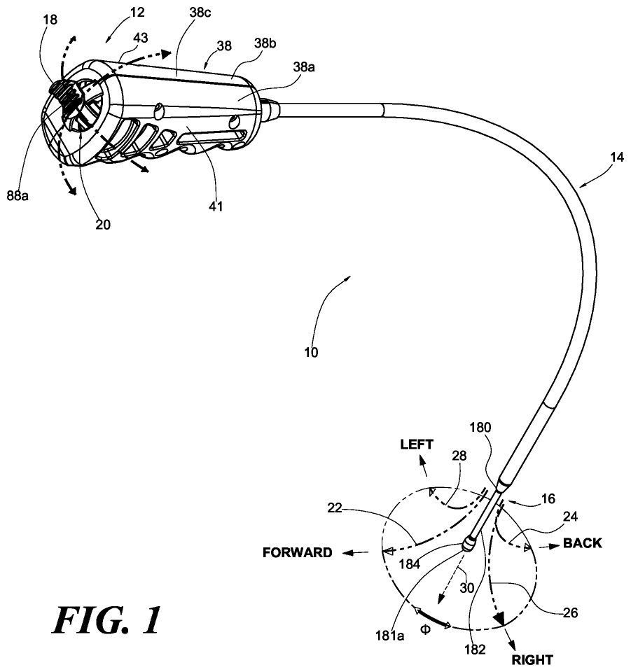

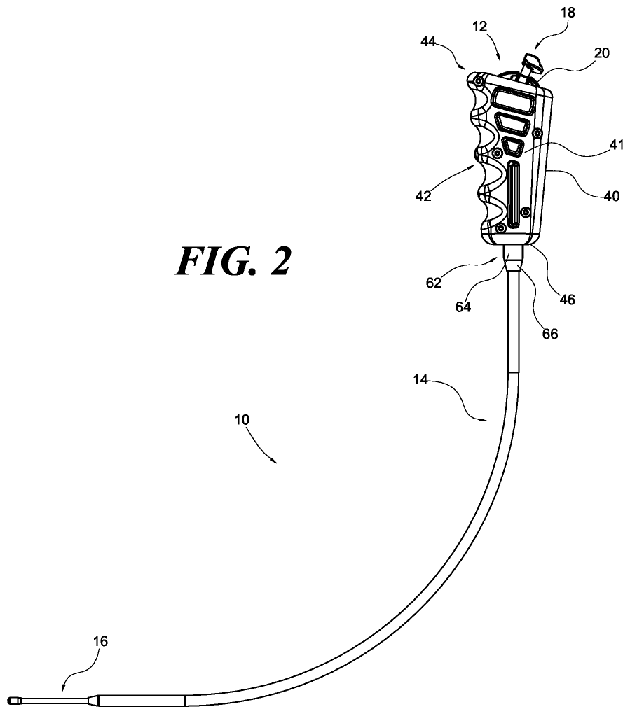

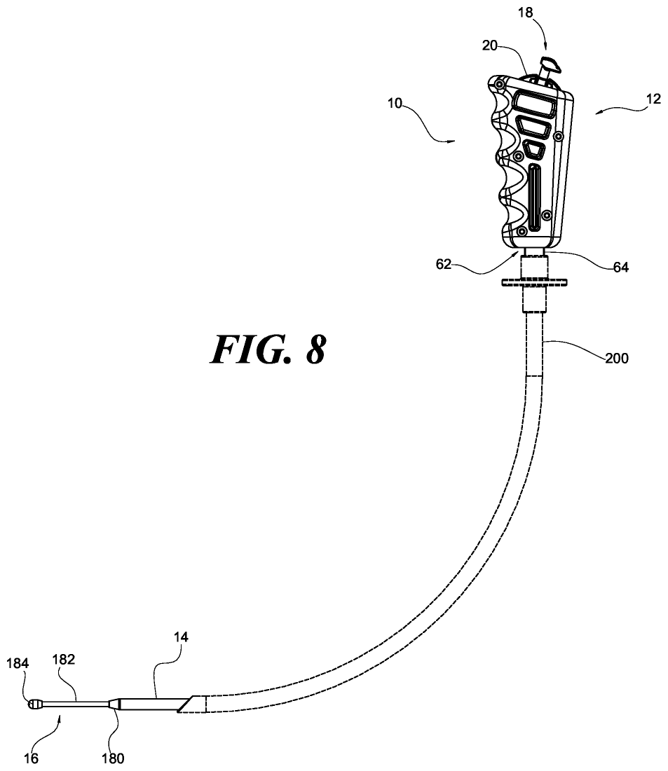

FIG. 1 shows a perspective view of a first embodiment of an articulable stylet or introducer 10. FIG. 2 shows a side plan view of the introducer 10. As will be discussed further below, the introducer 10 is used in an intubation procedure to place an endotracheal tube through the mouth and into the glottic opening of a patient. By way of example, FIG. 8 shows an endotracheal tube 200 disposed over the introducer 10, ready for oral insertion into the patient. In general, the introducer 10 and tube 200 are advanced through the mouth into the glottic opening, and then the introducer 10 is pulled out, leaving the endotracheal tube 200 in place. Ventilation equipment, not shown, can then be attached to the endotracheal tube 200 to assist the patient in ventilation.

With reference to FIGS. 1 and 2, a first embodiment is an articulable introducer 10 that includes a controller 12, a tube guide 14 and a flexible tip 16. In general, the controller 12 includes, a digitally manipulatable actuator 18, a rotatable bearing 20 and a handle 38. The actuator 18 is fixedly coupled to move the rotatable bearing 20. As will be discussed in further detail below, the handle 38 supports the bearing 20 such that the bearing can move in at least two degrees of freedom or at least two dimensions with respect to the handle 38 responsive to corresponding movement of the actuator 18.

The bearing 20 is operably coupled to the flexible tip 16 such that the flexible tip 16 moves in a way that corresponds to the rotatable movement of the bearing 20. Because the actuator 18 is fixedly coupled to the bearing 20, a user may manipulate the position of the flexible tip via movement of the actuator 18. In this manner, the actuator acts like a joystick. The unactuated position of the flexible tip 16 is in the axially outward direction from the end of the tube 14. This position is referred to as the rest position or center position 30.

In this embodiment, the relative movement of the flexible tip 16 with respect to the tube guide 14 (or the center/rest position 30) is substantially spherical in nature. In a spherical coordinate system (r, θ, φ), flexible tip 16 is movable in the θ (longitude) dimension and in the φ (latitude) dimension. With reference to FIG. 1, the flexible tip 16 is controllably movable or articulable (relative to the tube guide 14 or center/rest position 30) in a 360 degree range in the φ dimension, thus allowing the user to move the tip 16 in the forward direction 22, the backward direction 24, the right direction 26 and the left direction 28, as well as all angular directions in between.

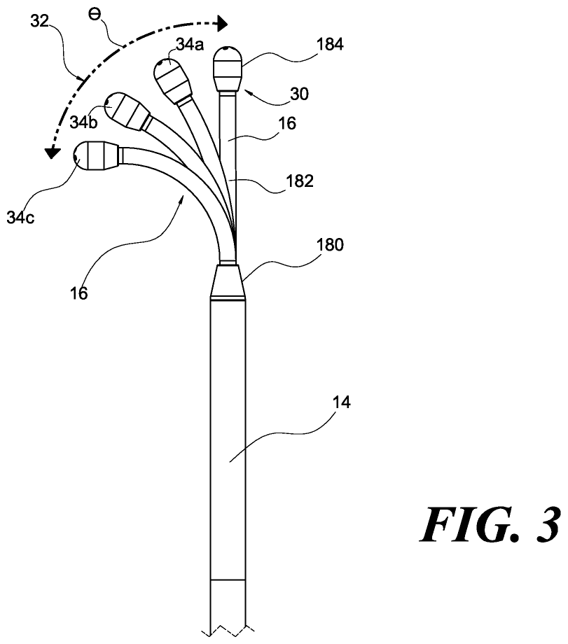

FIG. 3 shows the movement of the flexible tip 16 in the θ dimension, which has a range 32 of approximately 90 degrees. In particular, FIG. 3 shows a fragmentary perspective view of the tube guide 14 and flexible tip 16 illustrating the movement in the θ dimension of the flexible tip 16 from center position 30. In particular, FIG. 3 shows four exemplary positions 30, 34a, 34b, 34c of the flexible tip 16, including the center position 30, and three positions 34a, 34b, 34c having different non-zero angled in the θ direction.

As a consequence, the user may use the actuator 18 to bend (movement in the θ direction) in the forward direction 22, the backward direction 24, the right direction 26 and the left direction 28, as well as angular directions therebetween (φ angle).

It will be appreciated that the at least some advantages described herein can be achieved if the range of latitudinal position is less than 360 degrees, and/or of the range of longitudinal movement is less than 90 degrees. Indeed, at least some embodiments will have a range of longitudinal movement (i.e. bend angle) closer to 45 degrees. Having the ability to manipulate the tip to any set of multiple latitudinal (i.e. φ) positions and longitudinal positions (i.e. θ) provides at least some of the advantages discussed below. Again, from the user's perspective, this allows the tip 16 to bend (θ movement) in more than one left-right-forward-back (φ) direction. It will further be appreciated that the bending motion need not be spherical, but may take other shapes. As long as the tip 16 articulates in multiple left-right-forward-back (φ) directions, the user gains advantages.

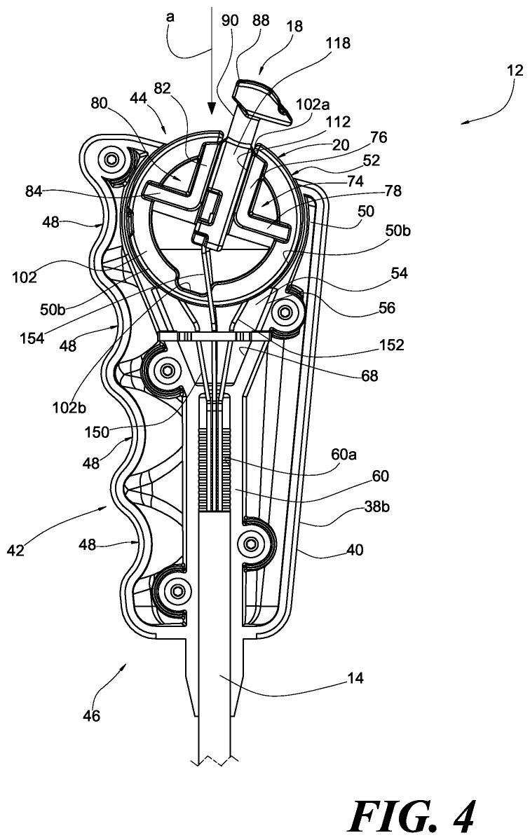

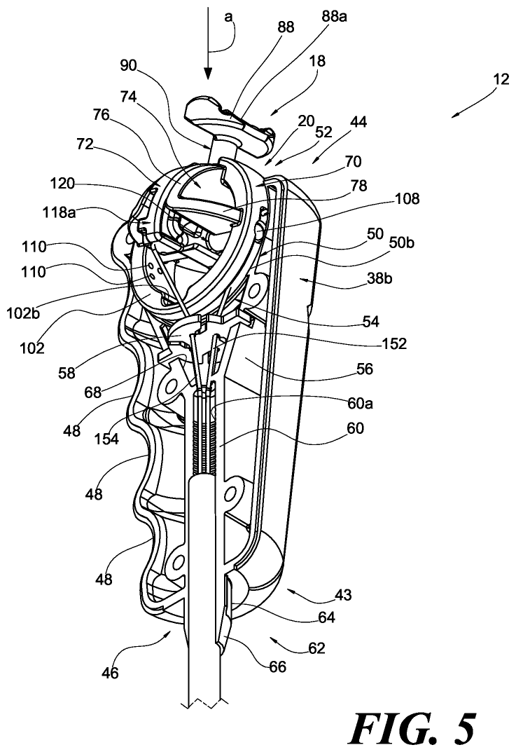

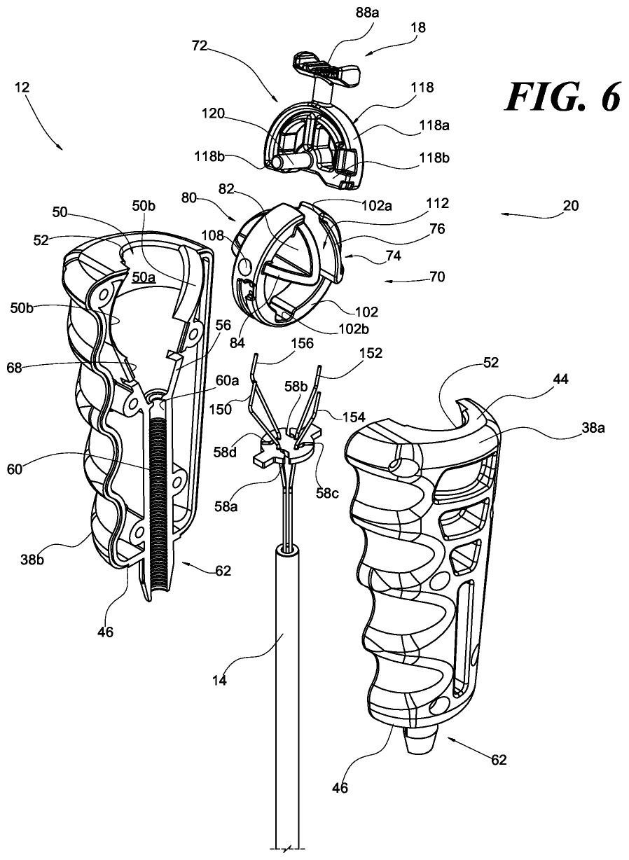

Referring again to the general structure of the introducer 10, FIGS. 4, 5 and 6 shows the controller 12 in further detail, with a fragmentary portion of the tube guide 14. FIG. 4 shows a side plan view of the controller 12 with one of the covers removed, FIG. 5 shows a perspective view of the controller 12 with one of the side covers removed, and FIG. 6 shows an exploded perspective view of the controller 12.

Referring to FIGS. 1, 4, 5 and 6, the handle 38 includes a first cover 38a and a second cover 38b joined a peripheral central seam 38c. The first cover 38a and second cover 38b have designs that a largely symmetrical to each other along the same 38c. The handle 38 has a back side 40, a left side 41, a front side 42, a right side 43, a top side 44 and a bottom 46. Each of the first cover 38a and the second cover 38b forms about half of each of the sides 40, 42, 44 and the bottom 44. The first cover 38a includes the left side 41 and the second cover 38b includes the right side 43.

The back side 40, the left side 41, the front side 42, and the right side 43 form a handle structure that extends from the top side 44 to the bottom 46, and which is configured to receive an adult human grip. The front side 42 preferably has finger grooves 48 formed therein, to facilitate a stable grip with a free thumb for manipulating the actuator 18, which extends from the top side 44. In this embodiment, the back side 40, the front side 42, the top side 44 and the bottom 46 are largely symmetrical about the intersection seam 38c of the two covers 38a, 38b, which allows for use by in either the right hand or the left hand. The handle 38 defines a vertical axis α that is intermediate back side 40, the left side 41, the front side 42, and the right side 43.

As shown in FIG. 6, the cover 38a and cover 38b are coupled by a plurality of fasteners 38d. In FIGS. 4 and 5, the cover 38a has been removed to reveal the interior structure of the cover 38b. It will be appreciated that the cover 38a has a mirror image of the interior structure of the cover 38b. With reference to FIGS. 5, 6 and 7, the cover 38 includes a bearing seat 50, a funnel section 56, and a tube seat 60.

The bearing seat 50 is configured to receive and retain the bearing 20 such that the bearing 20 may rotatable move in multiple degrees of freedom. To this end the bearing seat 50 in this embodiment has an interior surface 50a that defines a portion of a sphere and includes vertical channels 50b. In general, the bearing seat 50 retains the bearing 20 and allows partial spherical movement of the bearing 20. The bearing seat 50 has an open top 52 and an open bottom 54, and is centered about the common axis α. The open top 52 in this embodiment also forms an opening in the top side 44, and is concentric with the common axis α. The open bottom 54 opens into the funnel section 56.

The funnel section 56 extends from the top section open bottom 54 of the bearing seat 50 to the top end of the tube seat 60. The funnel section 56 defines a frustoconical void 68 having an axis on the common axis α. The frustoconical void 68 has a first diameter adjacent the open bottom 54 and converges to its narrow diameter at the top of the tube seat 60. It will be appreciated that, as with other structures in the interior of the handle 38, the cone section 56 and the corresponding section of the second cover 38b collectively form the entire funnel section 56 that defines the frustoconical void 68.

As shown in FIGS. 5 and 6, the controller 12 further includes a concentrator plate 58 having four slots 58a-58d. The concentrator plate 58 is in the form of a disk in this embodiment. The four slots 58a-58d extend inward from an outer periphery and terminate at locations equidistant from each other, and equidistant from the common axis α. The slots 58a-58d are configured to receive and guide the wire segments as discussed below. The concentrator plate 58 is received in and secured by features of the second cover 38b (and first cover 38a) within the funnel section 56, perpendicular to the common axis α. The inward extents of the four slots 58a-58d are within the frustoconical section 68. It will be appreciated that the slots 58a-58d may be replaced by corresponding throughholes which would also guide the wire segments.

As shown in FIGS. 4, 5 and 6, the tube seat 60 extends approximately from the bottom 46 to about halfway to the top side 44 terminating at the bottom of the funnel section 56. The tube seat 60 generally defines a cylindrical interior surface 60a that is configured to receive the tube guide 14, and is concentric with the common axis α. Thus, the center of the bearing seat 50, the axis of the frustoconical void 68 and the central axis of the cylindrical interior surface 60a are axially aligned along the common axis α.

The bearing 20 in this embodiment includes a fore-aft yoke 70 and a lateral yoke 72. The fore-aft yoke 70 includes an outer ring 102 and interior guides 74, 80. The outer ring 102 is partially received and retained with in the channel 50b of the bearing 50. As a consequence, the fore-aft yoke 72 can only rotate fore and aft, constrained by the channel 50b. The outer ring 102 further comprises hub bores 108 and a plurality of wire holes 110. The hub bores 108 extend radially through opposite sides of the outer ring 102 at about the vertical midpoint of the outer ring 102. As will be discussed further below, the hub bores 108 receive the axle 120 of the lateral yoke 72. The plurality of wire holes 110 are disposed below the hub bores, and are configured to receive the ends of wires therethrough. The outer ring 102 has a discontinuity at its top, forming a void 102a for receiving the actuator 18 and allowing movement of the actuator 18, as will discussed below. The bottom of the outer ring 102 includes a transverse inner groove 102b.

The guides 74, 80 cooperate to form a guide channel 112 for receiving a portion of the lateral yoke 72, as will be discussed below. The guides 74, 80 further cooperate to form a shroud to inhibit visual or physical access to the interior of the handle 38 via the open top 52. The guide 74 includes an arcuate vertical plate 76 and an arcuate lateral plate 78 that are joined at a common edge 75. The guide 80 similarly includes an arcuate vertical plate 82 and an arcuate lateral plate 84.

The arcuate vertical plates 76, 82 are oriented perpendicular to the circumference of the outer ring 102, and extend in a parallel and arcuate manner from opposite sides of the void 102a to locations approximately even with the vertical midlevel of the outer ring 102. Thus, the arcuate vertical plates 76, 82 and the void 102a form the guide channel 112. The arcuate lateral plates 78, 84 are likewise oriented perpendicular to the circumference of the outer ring 102, and are also oriented perpendicular to the vertical plates 76, 82.

Each of the arcuate lateral plates 78, 84 extends arcuately from approximately the vertical midpoint of the outer ring 102 to the bottom of the respective one of the vertical plates 76, 82 at the bottom of the guide channel 112. It will be appreciated that the radial extent of each of the plates 76, 78, 82, 84 is short of the radial extent of the outer ring 102, so as to allow the outermost portion of the outer ring 102 to seat within the channel 50b within the bearing seat 50 without interference from the plates 76, 78, 82, 84.

The lateral yoke 72 includes a half wheel 118, an axle 120. In this embodiment, the lateral yoke 72 is integrally formed with the actuator 18, forming the rigid connection between the bearing 20 and the actuator 18. The actuator 18 includes an engagement plate 88 and a connection post 90. The engagement plate 88 has a top surface 88a (see FIG. 1) that is configured to receive an adult human thumb, and may include a convex shape and surface features for friction. The connection post 90 extends from the bottom surface of the plate 88 to the outer surface of the top of the half wheel 118.

The half wheel 118 has a rim 118a, radial spokes 118b, and a hub 118c. The half wheel 118 has the general design of a spoked wheel that has had its bottom half removed. Thus, the rim 118a extends about 180 degrees about an axis on which the hub 118c is centered. Two of the radial spokes 118b form the bottom, with the hub 118c disposed therebetween. The rim 118a has an outer diameter configured to fit and rotate within the bearing seat 50 and guide channel 112. The half wheel 118 has an axial width sized to fit (and rotate) within the guide channel 112, and the transverse inner groove 102b. The axle 120 extends axially through the hub 118c and is rotatably received by the hub bores 108 in the fore-aft yoke 70.

In the rest or default position, shown in FIGS. 1, 4, and 5, the connection post 90 extends slight askew (least 10 to 30 degrees) of the common axis α. The engagement plate 88 is disposed perpendicular to the connection post 90, and thus tilts slightly backward toward the back side 40. This rest position corresponds to the neutral or center position 30 of the flexible tip 16. The slight backward tilt of the top surface 88a of engagement plate 88 and post 90 in the rest position provides improved ergonomics as compared to being strictly aligned along the common axis α.

The yokes 70 and 72 allow for movement of the actuator 18 any of the 360 degrees of position. To this end, the fore-aft yoke 70 allows rotation along the forward and backward dimension, and the yoke 72 allows rotation along lateral dimension, such that combinations of rotations can result in any angle in between. More specifically, the fore-aft yoke 70 is constrained to forward and backward travel by the vertical channels 50b, and the lateral yolk 72 is constrained to bidirectional lateral travel by the guide channel 112, and the transverse inner groove 102b. However, as the yoke 70 rotates, the axle 120 and constraints on the yoke 72 change, such full two-dimensional displacement of the actuator 18 is possible.

The two dimensional displacement of the actuator 18 and hence the bearing 20 is translated to the flexible tip 16 via four wire segments 150, 152, 154 and 156. The four wire segments 150, 152, 154 and 156 are operably coupled to the bearing 20 and to the flexible tip 16 to translate the movement of the actuator 18 and bearing 20 to the flexible tip 16. To this end, two of the wire segments 150, 152 are coupled to opposite sides of the outer ring 102 of the fore-aft yoke 70, and two of the wire segments 154, 156 are operably coupled to the opposite sides of the rim 118a. More specifically, the wire segments 150, 152 are coupled to respective locations adjacent to and just below the hub bore 108 on the outer ring 102 (via openings 110). The wire segments 154, 156 are coupled adjacent to respective radial spokes 118b of the half wheel 118. It will be appreciated that the wire segments 150, 152, 154 and 156 could be replaced by any other suitable flexible cord.

The wire segments 150, 152, 154 and 156 extend through corresponding slots 58a-58d in the concentrator plate 58, and then extend into the hollow interior of the tube guide 104. The wire segments 150, 152, 154 and 156 are then operably coupled to the flexible tip 16. In particular, as shown in FIGS. 1 to 3, the flexible tip 16 includes a coupler 180, a catheter 182, and a bulbous nub 184. The wire segments 150, 152 in this embodiment form a single continuous wire having ends connected to the fore-aft yoke 70 as described above, and wire body that wraps around the face 184a of the nub 184. Similarly, the wire segments 154, 156 in this embodiment form a single continuous wire having ends connected to the lateral yoke 72 as described above, and wire body that also wraps around the face 184a of the nub 184.

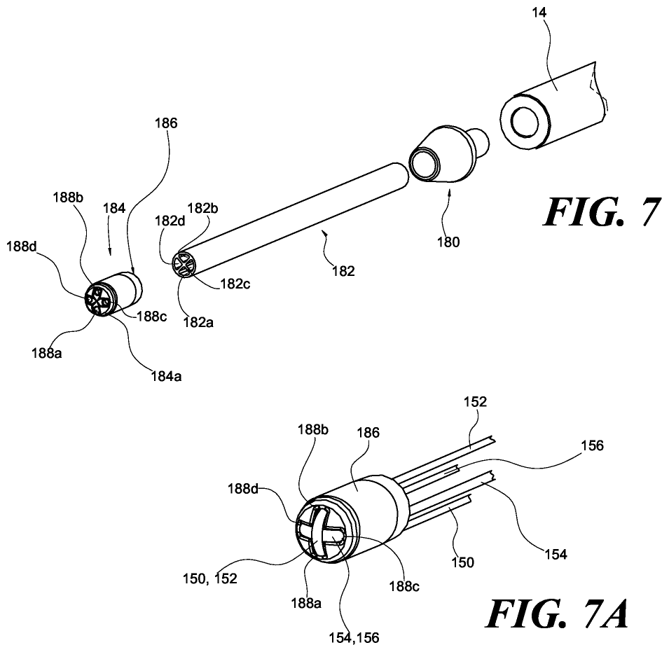

FIG. 7 shows an exploded view of an exemplary embodiment of the flexible tip 16 in further detail. The catheter 182 forms the flexible tubular member that moves in two degrees of freedom responsive to the movement of the actuator. The catheter 182 in this embodiment is quad-lumen catheter, having separate passages 182a-182d for each of the wire segments 150, 152, 154, 156, not shown in FIG. 7. The coupler 180 is a polymer device that couples the larger diameter tube guide 14 to the catheter 180, allowing the wire segments to pass therebetween. The bulbous nub 184 is affixed to the distal end of the catheter 182, and includes a body 186 having a hollow interior, and end face 184a having four openings 188a-188d. The openings 188a-188d preferably align with passages 182-182d of the catheter.

Although the wire segments 150, 152, 154 and 156 are omitted from FIG. 7 for clarity of exposition, FIG. 7A shows a fragmentary perspective view of the nub 184 with the wire segments 150, 152, 154 and 156 assembled thereto. The positions of the wire segments 150, 152, 154 and 156 is described with contemporaneous reference to FIGS. 7 and 7A.

The wire segment 150, not shown in FIG. 7, extends through the tube guide 16, the coupler 180, the passage 182a of the catheter 182, and through the bulbous nub 184. As shown in FIG. 7A, the wire segment 150 exits out of the opening 188a, and ends on the end face 184a. On the end face 184a, the wire is essentially both wire segments 150 and 152. As also shown in FIG. 7A, the wire segment 152 extends into the opening 188b. The wire segment 152 further extends through the bulbous nub 184, through the passage 182b of the catheter 182, through the coupler 180, and through tube guide 16.

In the same fashion, the wire segment 154, not shown in FIG. 7, extends through the tube guide 16, the coupler 180, the passage 182c of the catheter 182, and through the bulbous nub 184. As shown in FIG. 7A, the wire segment 154 exits out of the opening 188c, and ends on the end face 184a. On the end face 184a, the wire is essentially both wire segments 154 and 156. The wire segment 156 extends into the opening 188d, through the bulbous nub 184, through the passage 182d of the catheter 182, through the coupler 180, and through tube guide 16. It will be appreciated that in other embodiments, the wire segments 150, 152, 154, 156 may be formed of separate wires, each having ends connected to the actuator 20 and to the bulbous nub 184.

In any event, the wire segments the wire segments 150, 152, 154, 156 are relatively taut between the tip 16 and the rotatable bearing 20 when the actuator 18 is in the neutral (e.g. center) position. The ends of the wire segments 150, 152, 154, 156 should be sufficiently engaged at the tip 16 to prevent full sliding movement between the opposing segments of the same wire.

In operation, movement of actuator 18 causes rotation of the rotatable bearing 20. As discussed above, the yokes 70, 72 are operably coupled to allow two dimensional rotation of the rotatable bearing. Rotation of the rotatable member 20 pulls on one or two of the wire segments 150, 152, 154, 156 located opposite the direction of the actuator pull. The one or two pulled segments 150, 152, 154, 156 pull at the flexible tip from one direction corresponding to the direction of the pull, thereby causing the flexible tip 16 to flex toward the tension created by the pull. In this manner, movement of the actuator 18 in any combination of front, back, left or right movements creates a corresponding movement in the flexible tip 16 such as those described above in connection with FIGS. 1, 2 and 3.

Referring again to FIGS. 4, 5, 6 and 7, the handle 38 further includes a retainer 62 extending along the common axis externally away from the bottom 46. The retainer 62 facilitates retention of the endotracheal tube on the introducer 10 during the intubation procedure. The retainer 62 includes a cylindrical portion 64 that aligns with, connects to, and essentially forms a continuation of the tube seat 60. The retainer 62 also includes a frustoconical portion 66 that extends from the cylindrical portion 64. In general, the retainer 62 is sized and configured to receive an endotracheal tube, not shown in FIGS. 4 to 7, in a friction fit. The tube seat 60, the cylindrical portion 64, and the frustoconical portion 66 are aligned to provide a passage for the operative end of the guide tube 14. As discussed above, the distal end of the guide tube 14 connects to the flexible tip 16. In this embodiment, the tube guide 14 is hollow, and has a degree of flexibility.

In general, the tube guide member 14 and the flexible tip 16 are sized to slidingly receive an endotracheal tube which is commonly used for breathing assistance during anesthesia. To this end, FIG. 8 shows an endotracheal tube 200 disposed over tube guide 14, and engaged with the retainer 62. In this position, the user may insert flexible tip 16, followed by the tube guide 14 and tube 200, into the mouth of the patient. The user then can use the actuator 18 to manipulate the position of the tip 16 to help direct the tube guide 14 and tube 200 to the glottic opening of the patient not shown. After the tube 200 is in the proper position, the user may pull the tube 200 off of the connector tip 62 and slide the tube guide 14 and tip 16 out through the tube 200 while the tube remains in place. Once the tube guide 14 and tip 16 are removed from the tube 200, the operative end of the tube 200 may be connected to assisted respiration equipment.

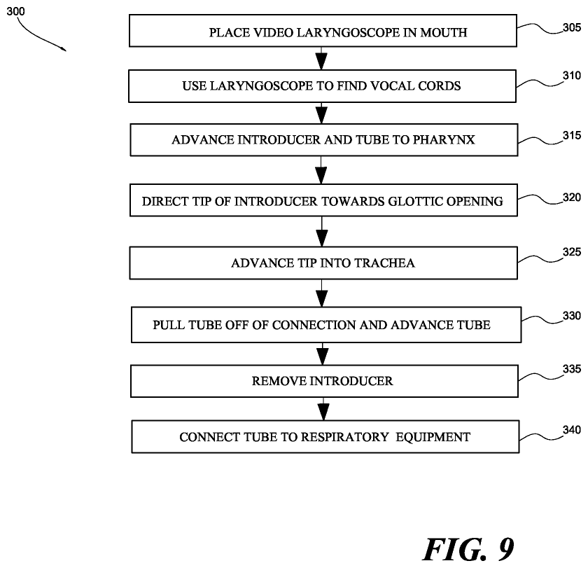

A more specific intubation procedure 300 is described herebelow in connection with FIG. 9. In step 305, a video laryngoscope, not shown, but which is known in the art, is placed in the mouth of the patient. In step 310, the physician uses the video laryngoscope to find a view of the vocal cords of the patient. In step 315, an assistant may then hold the video laryngoscope in place while the physician advances the introducer (i.e. stylet) 10 with a preloaded endotracheal tube 200, as shown in FIG. 8, into the mouth and to the posterior pharynx.

Thereafter in step 320, the physician then uses the actuator 18, which in this embodiment is thumb-controlled, to direct the tip 16 of stylet 10 in any direction 22, 24, 26, 28 or directions intermediate thereof to facilitate movement of the tip 16 towards the glottic opening (vocal cords). Once flexible tip 16 is at the glottic opening, the physician in step 325 advances the introducer 10 through opening into the trachea of the patient.

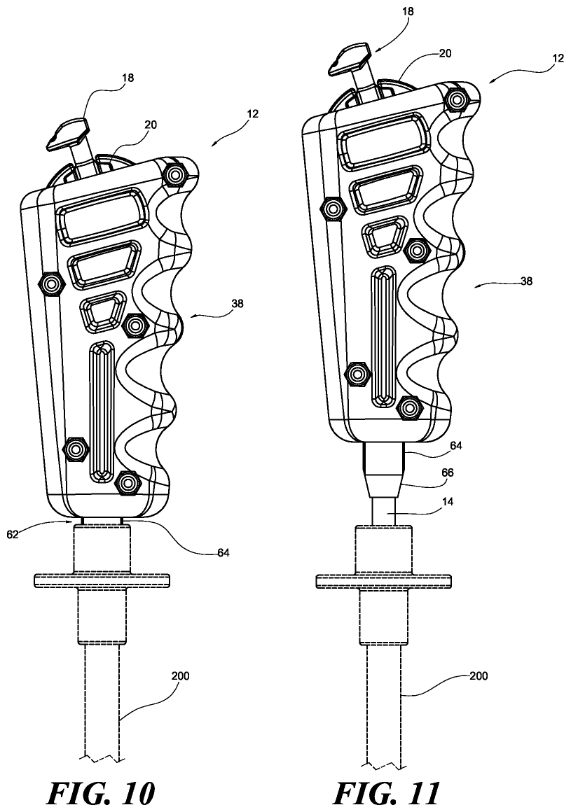

The physician then, step 330, pulls the endotracheal tube 200 off of the retainer 62. FIGS. 10 and 11 show fragmentary views of the endotracheal tube 200 and introducer 10. FIG. 10 shows the endotracheal tube 200 affixed to the introducer 10, and FIG. 11 shows the operative end of the endotracheal tube 200 taken off the retainer 62 in the process of separation of step 330. The endotracheal tube 200 may then be advanced along the guide 14 and over the tip 16 through the glottic opening into the trachea.

Thereafter, in step 335, the introducer 10 may then be removed. To this end, the user slides the introducer 10 back up through the operative end of the endotracheal tube 200. In step 340, the endotracheal tube 200 is connected to standard respiratory assistance equipment, which is known on the art.

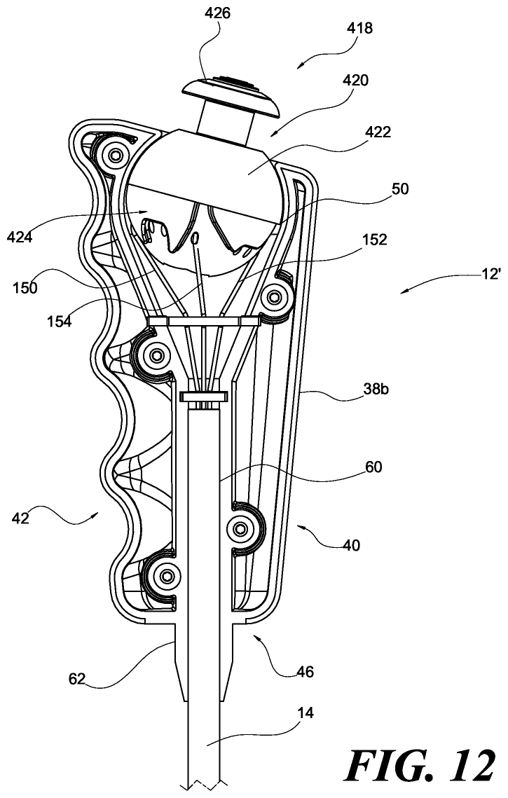

FIG. 12 shows a plan view of an alternative embodiment of the controller 12′. Specifically, the controller includes the handle 38 of FIGS. 4 to 6, an alternative actuator, and an alternative bearing 420. FIG. 12 shows the controller 12′ with the cover 38a removed, similar to the view of the controller 12 in FIG. 4. The controller 12′ may suitably be used with the tube 14 and flexible tip 16 in place of the controller 12.

In this embodiment, the dual yoke configuration of the bearing 20 has been replaced by a ball configuration. The bearing 420 is substantially a ball shape having the upper part 422 of its outer surface forming a sphere section that sized and configured to be retained by, and rotate within, the bearing seat 50. The bottom part 424 of the ball may be slightly smaller to better accommodate attachment of the wire sections 150, 152, 154 and 156 thereto. The actuator 418 in this embodiment has a ribbed, dome-shaped upper surface 426, but may otherwise have a similar structure as the actuator 18.

The simplicity of design of the ball shaped bearing 420 is an advantage, but the lack of rotational constraint (other than the wire tension) may be a disadvantage compared to the bearing 20. The choice of embodiment will depend on factors of the user's specific implementation.

The above-described embodiments of the introducer 10 are portable, and can be used in an operating room, ambulance, or a battlefield hospital. The tube introducer 10 can be provided as a disposable kit that includes the introducer 10 with a preloaded endotracheal tube 200. The handle 38 may suitably be constructed from injection molded polymer, and the tube guide 14 formed of a partly flexible polymer tube.

It will be appreciated that the above-described embodiments are merely exemplary, and that those of ordinary skill in the art may readily devise their own implementations and modifications that incorporate the principles of the present invention and fall within the spirit and scope thereof.

What is claimed is:

1. An endotracheal tube introducer, comprising
a controller having an actuator and a rotatable bearing, the actuator operably coupled to move the rotatable bearing;
a flexible tip;
a tube guide operably coupled between the controller and the flexible tip, wherein the flexible tip operably coupled to the rotatable bearing to move in at least two degrees of freedom responsive to movement of the actuator.

2. The introducer of claim 1, wherein the actuator is rigidly coupled to the rotatable bearing and is digitally manipulatable to rotate the rotatable bearing in two degrees of freedom.

3. The introducer of claim 2, further comprising flexible cord segments coupled to the rotatable bearing and the flexible tip, the flexible cord segments operably coupled to translate movement of the rotatable bearing to the flexible tip.

4. The introducer of claim 3, wherein the flexible cord segments pass through an interior of the tube guide.

5. The introducer of claim 4, wherein the flexible tip has one or more lumens for receiving the flexible cord segments therethrough.

6. The introducer of claim 1, further comprising a retainer configured to removably secure the endotracheal tube over the tube guide.

7. The introducer of claim 6, wherein the retainer includes a frustoconical portion and is configured to friction fit the endotracheal tub.

8. The introducer of claim 1, wherein the controller further comprises a handle, the handle a seat receiving the rotatable bearing.

9. The introducer of claim 8, wherein the handle defines an opening for actuator, said opening defining limits of a partial spherical motion range of motion of the actuator.

10. An endotracheal tube introducer, comprising
a controller having an actuator and a rotatable bearing, the actuator operably coupled to move the rotatable bearing;
a tube guide coupled to the controller;
a flexible tip coupled to the tube guide and having a unactuated first position, the flexible tip further operably coupled to the rotatable bearing to articulate in at least two different directions from the unactuated first position responsive to movement of the actuator.

11. The introducer of claim 10, further comprising a plurality of flexible cord segments operably coupled between rotatable bearing and the flexible tip to translate movement of the rotatable bearing to the flexible tip.

12. The introducer of claim 11, wherein the flexible cord segments comprise wire segments.

13. The introducer of claim 11, wherein the controller further comprises a bearing seat, and wherein the rotatable bearing further comprises a first yoke and a second yoke, the first yoke rotatable about an axis with respect to the bearing seat, the second yoke rotatable about an axis with respect to the first yoke, and wherein at least a first flexible cord segment is coupled to the first yoke, and at least a second flexible cord segment is coupled to the second yoke.

14. The introducer of claim 13, wherein the first yoke includes an outer ring configured to be movably received within a channel defined in the bearing seat.

15. The introducer of claim 14, wherein the outer ring includes a channel configured to movably receive a rotation portion of the second yoke.

16. The introducer of claim 11, wherein the flexible cord segments pass through an interior of the tube guide from the actuator to the flexible tip.

17. The introducer of claim 16, wherein the flexible tip includes a quad-lumen catheter operably coupled to the tube guide at one end, and a nob affixed to a second end of the tube guide, and wherein each of the flexible cord segments passes through a corresponding passage in the quad-lumen catheter and is operably coupled to the nob.

18. The introducer of claim 9, wherein the flexible tip is further operably coupled to the rotatable bearing to articulate in different directions having a range of 360 degrees from the unactuated first position responsive to movement of the actuator.
